(19) United States
(12) Patent Application Publication
(10) Pub. No.: US 2011/0224554 A1
(43) Pub. Date: Sep. 15, 2011

(54) Optogenetic Fiber Optic Cannula and Adapted Fiber Optic Connector

(75) Inventors: Mirko Vukeljic, Quebec (CA); Jean-Luc Neron, Quebec (CA); Sead Doric, L'Ancienne-Lorette (CA)

(73) Assignee: OPTOMAK INC., L'Ancienne-Lorette (CA)

(21) Appl. No.: 13/046,904

(22) Filed: Mar. 14, 2011

Related U.S. Application Data
(60) Provisional application No. 61/313,258, filed on Mar. 12, 2010.

(51) Int. Cl. A61B 6/00; G02B 6/38

(52) U.S. Cl. 600/478; 385/72

(57) ABSTRACT

A cannula can have a ferrule with two interspaced optical fiber passages extending therebetween, each securely housing an optical fiber therein having a first tip exposed at a connection end, and a second tip protruding from an opposite implant end by a penetration distance, and a bore extending into the ferrule from the connection end. The cannula can be removably connected by a patch cord having a ferrule with a guide pin, with a relatively high degree of optical alignment, by inserting both ferrules into corresponding ends of a sleeve and engaging the guide pin within the bore.

### CROSS-REFERENCE TO RELATED APPLICATIONS/PRIORITY CLAIM

This application claims priority of U.S. provisional application No. 61/313,258, filed Mar. 12, 2010 by applicant, the contents of which are hereby incorporated by reference.

### BACKGROUND

The field of optogenetics is a very promising sector of health sciences which receives much researching efforts. Essentially, many optogenetic research applications involve emitting light from an optical fiber into body tissue, typically of a mammal such as mice or rats. This typically involves adhering a housing of the optical fiber, referred to as a fiber optic cannula, to the surface of the body with optical fiber extending therefrom into the body tissue.

### SUMMARY

Some research applications done on neurons require implanting two or more optical fibers at precise locations in the brain of the animal. Given the small size of the fiber optic components, this was difficult to achieve.

It was found that a high degree of precision in the distance between two or more fibers could be achieved by housing such fibers in a common ferrule. However, because it is often required to disconnect the animal from the optical source between experiments, there is a specific need for a special connector which would allow connecting-disconnecting the cannula to/from the optical source, while allowing the cannula to have a small size so as to create as little discomfort to the animal between the experiments. This connector should allow for the precise angular alignment of the optical fibers in the cannula with the ones of the patch cord, to reduce power losses.

Further, it is sometimes required to provide liquids and/or electric wires or the like close to the location of the optical fibers in the tissue. It was found that this can be achieved by providing for a conduit passage extending through the cannula and connector.

In accordance with one aspect, there is provided a fiber-optic cannula comprising a ferrule with a central axis, a connection end and a implant end opposite the connection end and at least two interspaced optical fiber passages extending therebetween, parallel to said central axis and each securely housing an optical fiber therein having a first tip exposed at said connection end, and a second tip protruding from said implant end by a penetration distance, a guide axis parallel to and spaced from both the optical fiber passages and the central axis, and a bore extending into the ferrule from the connection end along the guide axis.

In accordance with another aspect, there is provided a fiber-optic connector comprising a first ferrule and a second ferrule, both ferules having a body portion having a straight external surface with a central axis, a connector end and a distal end and at least two interspaced optical fiber passages extending therebetween, parallel to said central axis and each securely housing an optical fiber therein having a tip exposed at said connector end, and a guide axis parallel to and spaced from both the optical fiber passages and the central axis; wherein the first ferrule has a male guide pin protruding from the connector end along the guide axis, and the second ferrule has female bore mating the male guide pin and extending inwardly from the connector end along the guide axis; and a sleeve having two opposite open ends and an internal surface mating with the external surface of both ferrules to slidingly receive each ferrule at a corresponding end with the guide pin received in the bore and the optical fiber tips being aligned with corresponding optical fiber tips of the other ferrule.

In accordance with another aspect, there is provided a cylindrically shaped fiber-optic cannula comprising: a cylindrical ferrule having a connection end with a mating face for releasable connection to a fiber-optic cord and an implanting end for insertion into tissue, said ferrule having: at least one fiber-optic channel therethrough with an optical fiber tightly mounted therein from said connection end to said implanting end for carrying light from said fiber-optic cord into a biological tissue; a cylindrical outside surface adapted for tight insertion in a cylindrical sleeve for axial alignment of said fiber-optic connector ferrule with a corresponding ferrule with mating faces toward one another, for connection of said optical fiber from said cylindrical ferrule to said fiber-optic cord; and an off-center guiding hole adapted to tightly receive a guiding rod continuously from said cylindrical fiber-optic connector ferrule to said corresponding ferrule for angular alignment thereof.

Many further features and combinations thereof concerning the present improvements will appear to those skilled in the art following a reading of the instant disclosure.

### DESCRIPTION OF THE FIGURES

In the figures,

FIG. 1 is a schematic view showing an optogenetic system for delivering light into an organism;

FIG. 2 is a view showing the fiber optic connector between a patch cord and cannula of the system shown in FIG. 1;

FIG. 3 is a front elevation of the cannula of FIG. 2;

FIGS. 4 and 5 are cross sectional views taken along cross-section lines 4-4 and 5-5 of FIG. 3.

### DETAILED DESCRIPTION

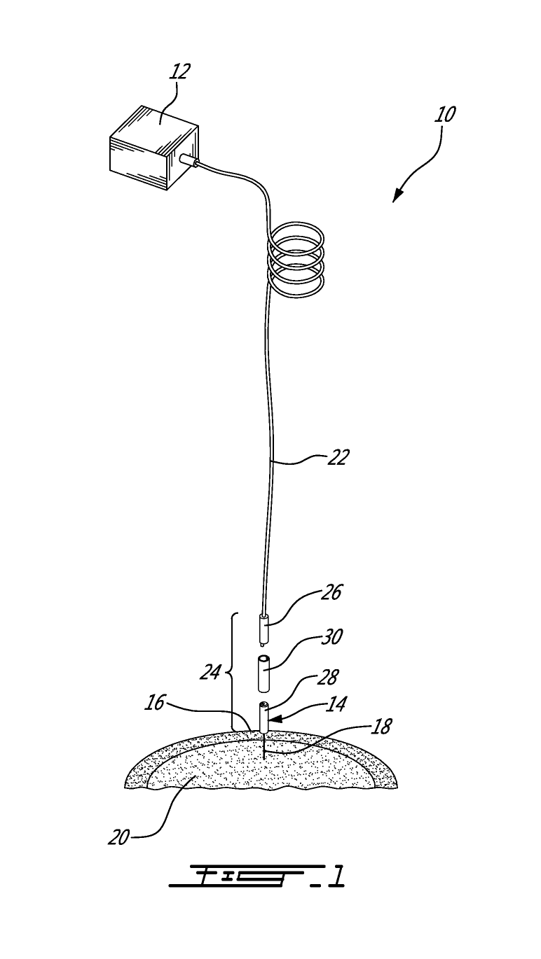

FIG. 1 shows an example of an optogenetic system 10. In this example, the optogenetic system 10 includes a light source 12 which can be a LED source, laser source, white light source for instance. At the other end, a fiber optic cannula 14 is shown adhered to a body surface 16 of a mammal, with optical fiber 18 penetrating into body tissue 20 of the mammal to provide light thereto. A patch cord 22 connects the light source to the cannula 14. The connector 24 between the patch cord 22 and the cannula 14 includes a ferrule 26 provided at the end of the patch cord 22, a ferrule 28 forming part of the cannula 14, and a sleeve 30 which can receive both ferrules 26, 28.

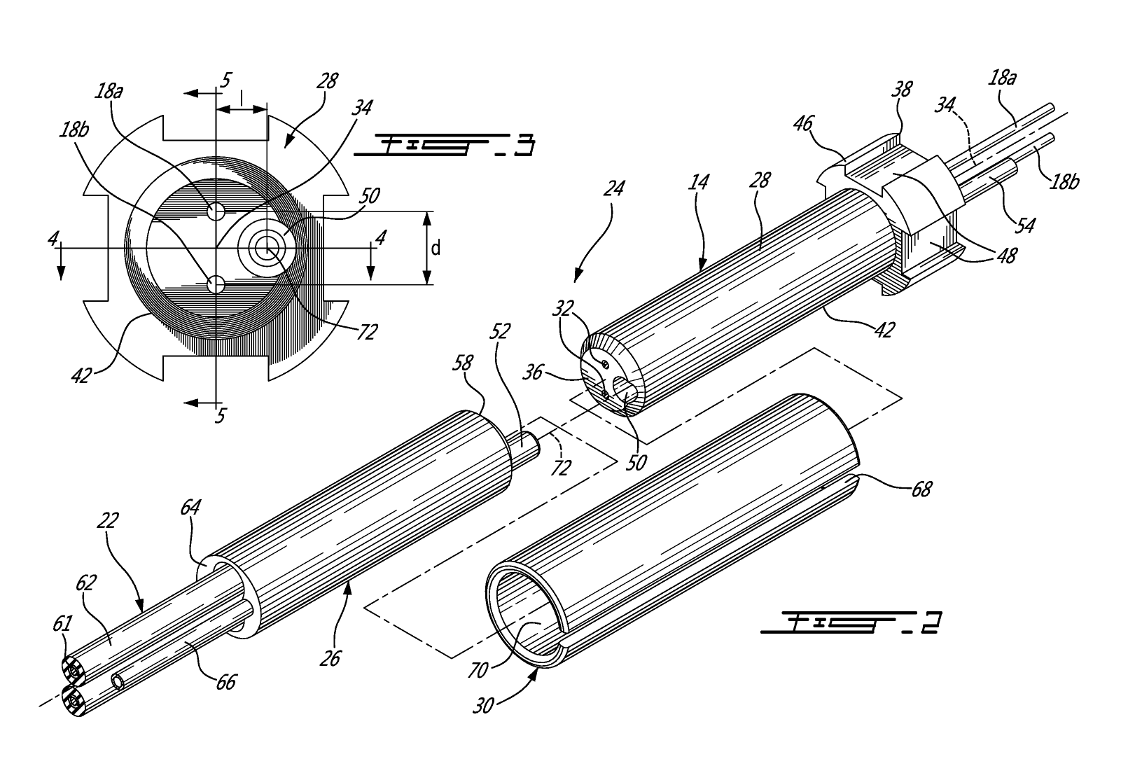

The components of the cannula 14 and connector 24 are shown in greater detail on FIG. 2. The patch cord 22 is shown on the left hand side and the cannula 14 is shown on the right hand side. In this particular example, the cannula 14 is a two-fiber cannula which houses two optical fibers 18a, 18b which can be interspaced from one another by a very precise distance (d, shown in FIG. 5). More precisely, the ferrule 28 has two optical fiber passages, which can be made by high precision machining of the ferrule 28 for instance. The optical fiber passages 32 can be seen to be parallel to a central axis 34 of the ferrule 28, and extend from a connection end 36 thereof (which is typically precisely polished flat) to a distal end, or implant end, adapted to attachment to a body surface. During use, the distal end 38 is adhered to the surface of the body. In this particular example, the ferrule 28 has an elongated body portion 42 having an external surface forming a sleeve engagement path and extending from the connection end 36, and a flange portion 46 at the distal end 38. Optical fibers 18a, 18b, better seen on FIG. 5, are housed in the optical fiber passages 32 and extend from the connection end to protrude outwardly from the distal end 38 by a penetration distance D.

Referring back to FIG. 2, it will be noted that the flange portion 46 is optional, and is used in this example with longitudinal grooves 48. The cannula 14 can be adhered to a surface of a body such as the skull of a mouse with the optical fibers 18a, 18b extending into body tissue, such as the brain, to stimulate neurons with light signals. The adhesion can be done with a thick adhesive substance. When longitudinal grooves 48 are used in the flange, the adhesive can set into the grooves 48 and the grooves 48 thus contribute to provide further rotation resistance to the adhered cannula 14.

As also shown in FIG. 5, the cannula 14 also has a bore 50 to act as a socket, i.e. delimiting a pin reception path, to receive a guide pin 52 of the patch cord 22 and contribute to maintain alignment of the optical fibers in the connection between the two ferrules 26, 28. In this specific embodiment, the cannula 14 also has an optional hollow needle 54, the use of which will be detailed below. In alternate embodiment, there can be more than two optical fiber passages, or just one, for instance.

For the cannula 14 not to be too cumbersome for the animal on which it is adhered, the ferrule 28 is preferably kept small. In this specific example, the ferrule 28 has an elongated body portion 42 having a circular cross-section (shown in FIG. 3). For exemplary purposes, it will be mentioned here that in this particular case the circular cross-section has a diameter of 2.5 mm and the length of the ferrule 28 can be between 6 an 10 mm, for example. Other dimensions are possible as well, and in alternate embodiments, the cross-section can be oval or elliptical for instance. Further in this example, optical fibers having a core diameter of 200 μm and a cladding diameter of 240 μm can be used for instance, and the optical fiber passages can be spaced apart from one another by a distance d such as between 0.5 and 2 mm. Other dimensions of optical fibers can be used as well, such as single mode having 9-10 μm core or multi-mode having a 500 μm core diameter, for instance.

In this example, the patch cord 22 has a ferrule 26 quite similar to the one used in the cannula 14. This ferrule 26 also has two interspaced optical fiber passages (54a, 54b in FIG. 5) interspaced from one another, and two associated optical fibers 56a, 56b precisely reaching the connection end 58 and having buffer 60 and jacket 62 shown extending out from the distal end 64. An optional tube 66 or jacket can also be used, the use of which will be discussed below. The elements extending out the distal end 64 can be covered by a sheathing, which is not shown in FIG. 2. Of course, the position of the optical fiber passages 54a, 54b in the cross-section of the body portion will be made to correspond closely to the position of the optical fiber passages 32 in the cross-section in the cannula (as shown in FIG. 3). Otherwise, the misalignment of at least one of the two optical fiber connections may be inevitable, and generate undesired power losses. One difference of the patch cord ferrule 26 is the absence of a flange portion 46, which was not required in this case. Another difference resides in the fact that this ferrule 26 has a guide pin 52 providing a male portion protruding from the connection end 58.

A sleeve 30 is provided which receives the body portions of both ferrules 26, 28 to maintain the connection. The tighter the fit between the sleeve 30 and the ferrules 26 and 28, and between the guide pin 52 and the bore 50, the firmer the connection will be held together. In this particular case, it will be noted that to achieve a very tight fit, the sleeve 30 is provided with a slit 68 along its entire length, and can be made of a material which is at least slightly elastic, such as zirconia, and is manufactured with an interference fit with the diameter of the body portion 42 of the ferrules 26, 28 it receives. Henceforth, when the ferrules 26, 28 which can have a bevelled connection end are pushed into a corresponding end of the sleeve 30, the ferrules 26, 28 push the slit 68 open, and the elasticity of the sleeve material thereafter biases the slit 68 to close it. The internal surface 70 of the sleeve thus exerts a force against the external surface 42 of the ferrules, which increases the amount of frictional resistance to disconnection. The amount of frictional resistance to disconnection is also affected by the area of contact between the sleeve 30 and ferrules 26, 28, and thus by the length and diameter of the ferrules 26, 28 and sleeve 30.

It is understood from the above that the tightness of the fit between the sleeve 30 and the ferrules 26, 28 is directly related to the precision obtainable with the alignment of the optical fiber connection. Another important factor is the position of the guide pin 52 and mating bore 50 in the cross section and the tightness of the fit between the guide pin 52 and bore 50. In fact, the guide pin 52 and bore 50 provide an angular alignment feature, and contribute to maintain the angular alignment when the connector 24 is subjected to torsion stress. To achieve good precision, both the guide pin position and bore position should precisely correspond to an axis referred to herein as the guiding axis 72, relative to the parallel central axis 34 and optical fiber passages. Further, it will be understood that positioning the guiding axis 72 further away from the central axis 34 will typically increase angular alignment precision because it increases the lever arm 1 with the central axis 34, or rotation axis. In this particular example, the guiding axis 72 is spaced by about 0.5 mm from the outer surface 42 of the ferrules 26, 28 which come into contact with the sleeve 30.

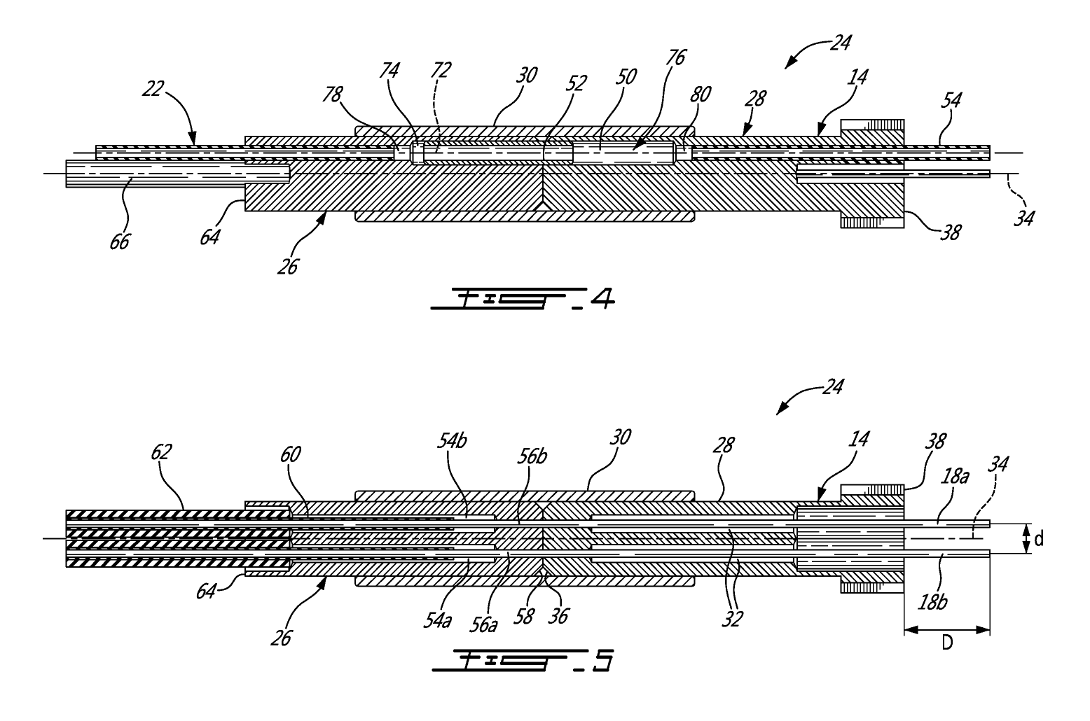

Turning now to FIG. 4, it will be seen that in this particular case, openings in both ferrules 26, 28 can be machined in exactly the same manner in a molded or machined body of material such as zirconia, metal or plastic for instance, and the guide pin 52 can be inserted into a bore 74 of one of the ferrules 26 and adhered into place for instance. In an alternate embodiment, the guide pin 52 can be provided in the cannula portion 14 of the connector 24 rather than in the patch cord portion 22 of the connector 24, though positioning it in the patch cord portion 22 can be preferred to reduce the weight and cumbersomeness of the cannula 14 for the animal when it is disconnected from the patch cord 22.

A further optional feature of the cannula 14 and connector 24 provided in this embodiment is also shown in FIG. 4. The optional feature is a conduit passage which can extend across and through both ferrules 26, 28 to deliver a fluid, electrical wires or the like through the cannula 14 and into the body tissue. The conduit passage 76 can include a passage portion 78, 80 extending further from the bottom of the bore 50, 74 of both ferrules 26, 28, and entirely across, and the guide pin 52 can hollow, so as to allow continuing the passage across it. If fluid is to be delivered, it can be provided into the patch cord ferrule 26 from a flexible tube 66 inserted securely into the conduit passage at the distal end 64. A hollow needle 54 can be provided at the implant end 38 of the cannula 14 to prolong the conduit passage 76 into the body tissue. Providing the conduit passage 76 through the guide pin 52 is particularly practical to deliver fluid. In this case, the guide pin 52 can serve both as a portion of the conduit passage 76 and an angular alignment feature for the optical connection.

It will be noted that alternate embodiments can have more than one guide pin and more than one conduit passage if desired.

As can be seen from the above, the examples described and illustrated are intended to be exemplary only. The scope is indicated by the appended claims.

What is claimed is:

1. A fiber-optic cannula comprising a ferrule with an external surface delimiting a sleeve engagement path, a central axis, a connection end and an implant end opposite the connection end and at least two interspaced optical fiber passages extending therebetween, parallel to said central axis and each securely housing an optical fiber therein having a first tip exposed at said connection end, and a second tip protruding from said implant end by a penetration distance, a guide axis parallel to and spaced from both the optical fiber passages and the central axis, and a bore extending into the ferrule from the connection end along the guide axis and delimiting a pin reception path.

2. The fiber-optic cannula of claim 1 wherein the implant end is adhered to a body surface of a mammal, with the optical fiber penetrating into body tissue of the mammal.

3. The fiber-optic cannula of claim 1 wherein the cannula has a flange portion at the implant end, the flange portion having a surface having a plurality of circumferentially interspaced and longitudinally aligned grooves.

4. The fiber-optic cannula of claim 1 wherein the connection end is polished flat.

5. The fiber-optic cannula of claim 1 wherein a conduit passage extends from the bore across the ferrule along the guide axis.

6. The fiber-optic cannula of claim 5 further comprising a hollow needle secured at the implant end and in fluid communication with the conduit passage.

7. The fiber-optic cannula of claim 1 wherein the ferrule has a body portion leading to the connection end, the body portion having a circular cross-section.

8. The fiber-optic cannula of claim 7 wherein the guide axis is closer to a cylindrical external face of the body portion than to the central axis.

9. A fiber-optic connector comprising a first ferrule and a second ferrule, both ferrules having a body portion having a straight external surface with a central axis, a connector end and a distal end and at least two interspaced optical fiber passages extending therebetween, parallel to said central axis and each securely housing an optical fiber therein having a tip exposed at said connector end, and a guide axis parallel to and spaced from both the optical fiber passages and the central axis; wherein the first ferrule has a male guide pin protruding from the connector end along the guide axis, and the second ferrule has female bore mating the male guide pin and extending inwardly from the connector end along the guide axis; and a sleeve having two opposite open ends and an internal surface mating with the external surface of both ferrules to slidingly receive each ferrule at a corresponding end with the guide pin received in the bore and the optical fiber tips being aligned with corresponding optical fiber tips of the other ferrule.

10. The fiber-optic connector of claim 9 wherein the guide pin is hollow and a conduit passage extends along the guide axis through both ferrules and the guide pin.

11. The fiber-optic connector of claim 10 wherein a tube is connected at the distal end of the first ferrule in fluid communication with the conduit passage.

12. The fiber-optic connector of claim 9 wherein the first ferrule has a female bore securely receiving the guide pin.

13. The fiber-optic connector of claim 12 wherein the guide pin is adhered in the female bore of the first ferrule with an adhesive.

14. The fiber-optic connector of claim 9 wherein the optical fibers are adhered in the respective optical fiber passages with an adhesive.

15. The fiber-optic connector of claim 9 wherein the first ferrule is provided at the tip of a fiber-optic patch cord.

16. The fiber-optic connector of claim 15 wherein the optical fibers of the second ferrule each have an other tip protruding from said distal end by a penetration distance, and the distal end is adherable to a body surface of a mammal, with the optical fiber penetrating into body tissue of the mammal.

17. The fiber-optic connector of claim 9 wherein the sleeve has a lengthwise slit defined therein and is formed of a material having elasticity to cause a closing spring action when the slit is broadened, wherein the internal surface of the sleeve forms an interference-fit with the external surface of both ferrules for the ferrules to broaden the slit when inserted therein and the spring action to thereafter hold the ferrules inside the sleeve.

18. The fiber-optic connector of claim 17 wherein the external surfaces of both ferrules and the internal surface of the sleeve are cylindrical.

19. A cylindrically shaped fiber-optic cannula comprising:
a cylindrical ferrule having a connection end with a mating face for releasable connection to a fiber-optic cord and an implanting end for insertion into tissue, said ferrule having:
at least one fiber-optic channel therethrough with an optical fiber tightly mounted therein from said connection end to said implanting end for carrying light from said fiber-optic cord into a biological tissue;
a cylindrical outside surface adapted for tight insertion in a cylindrical sleeve for axial alignment of said fiber-optic connector ferrule with a corresponding ferrule with mating faces toward one another, for connection of said optical fiber from said cylindrical ferrule to said fiber-optic cord; and
an off-center guiding hole adapted to tightly receive a guiding rod continuously from said cylindrical fiber-optic connector ferrule to said corresponding ferrule for angular alignment thereof.

20. The fiber-optic cannula as claimed in claim 19, wherein said guiding hole passes all the way through said cylindrical ferrule and said guiding rod is hollow to form a channel extending from said connection end to said implanting end.
